(19) United States
(12) Patent Application Publication
(10) Pub. No.: US 2026/0325554 A1
(43) Pub. Date: Oct. 22, 2026

(54) WATER-DISPERSIBLE CANNABINOID EMULSION FORMULATIONS, METHODS OF MAKING AND APPLICATIONS

(71) Applicant: 2682130 ONTARIO LIMITED, Tecumseh (CA)

(72) Inventors: Justin Frank BINDER, Windsor (CA); Abhinandan BANERJEE, Windsor (CA); Krunalkumar Kiritbhai PATEL, Windsor (CA); Ian SCOTT, Windsor (CA); John Frederick TRANT, LaSalle (CA)

(73) Assignee: 2682130 ONTARIO LIMITED, Tecumseh, ON (CA)

(21) Appl. No.: 19/106,820

(22) PCT Filed: Aug. 29, 2023
(86) PCT No.: PCT/IB2023/058536

§ 371 Date: Feb. 26, 2025

Related U.S. Application Data
(60) Provisional application No. 63/373,915, filed on Aug. 30, 2022.

(51) Int. Cl. A61K 31/00; A61K 9/107; A61K 47/26; A61K 47/44

(52) U.S. Cl. CPC A61K 31/658; A61K 9/107; A61K 47/26; A61K 47/44

(57) ABSTRACT

An example of a water dispersible emulsion is provided. The water dispersible emulsion includes a cannabinoid at about 0.1-5.0% (w/w) collectively. In addition, the water dispersible emulsion includes an edible oil at about 0.1-15.0% (w/w). Furthermore, the water dispersible emulsion includes an emulsifying agent at about 0.1-10.0% (w/w).

### FIELD

The present invention relates generally to water-dispersible emulsion formulations, and more particularly to water-dispersible emulsion formulations comprising cannabinoids or cannabis-derived compounds for use in beverages and other water-based or water-containing products.

### BACKGROUND

Cannabinoids refer to a group of chemicals that that can act on cannabinoid receptors. Cannabinoid receptor ligands include endocannabinoids, which can be found naturally occurring in humans and other animals, phytocannabinoids, which can be found in cannabis and other plants, other plants, and lichens, and synthetic cannabinoids. Cannabinoids also include tetrahydrocannabinol (THC), such as delta-9-tetrahydrocannabinol, and cannabidiol (CBD), which are known to be psychoactive.

Oral ingestion of cannabinoids or other cannabis-derived compounds for medical or recreational purposes is quickly becoming a popular consumption method. However, challenges with cannabinoid stability and oral bioavailability of cannabinoids exist with edible preparations, such as cannabinoid-infused oils and edibles. Being a highly lipophilic and essentially water insoluble, CBD and THC are difficult to formulate in relatively high concentration for consumption without increasing the oily fraction to be consumed. Emulsion and other colloidal formulations are sometimes used to overcome issues with oral bioavailability and colloidal stability for many lipophilic nutraceutical substances in water-based or water-containing preparations (such as curcumin, lutein, lycopene, vitamins A, D and E, coenzyme Q10, and others). However, issues with the chemical stability of the bioactive compounds, the colloidal stability of the emulsion formulations, and stability of the formulations towards microbial growth are difficult to overcome. For cannabinoids and other compounds found in cannabis, these issues are daunting-cannabinoids and many other compounds found in cannabis are highly susceptible to oxidation and other types of chemical degradation; they are highly viscous, resinous, and/or crystalline solids, which are difficult to disperse and homogenize; the input materials (like cannabis extracts or cannabis-derived oils) can often contain microbial contaminants due to being derived from materials of biological origin.

Due to these difficulties, there are very few commercially available infusion technologies that meet the criteria to produce cannabinoid-infused beverage products.

### BRIEF DESCRIPTION OF THE DRAWINGS

Reference will now be made, by way of example only, to the accompanying drawings in which:

FIG. 1A shows intensity weighted particle size distribution profiles of a cannabinoid comprising about 85% w/w THC distillate;

FIG. 1B shows an example of the volume weighted particle size distribution profiles of a cannabinoid comprising about 85% w/w THC distillate;

FIG. 2A shows intensity weighted particle size distribution profiles of a cannabinoid comprising about 80% w/w CBD distillate;

FIG. 2B shows an example of the volume weighted particle size distribution profiles of a cannabinoid comprising about 80% w/w CBD distillate;

FIG. 3A shows intensity weighted particle size distribution profiles of a cannabinoid comprising about 95% w/w CBD distillate;

FIG. 3B shows an example of the volume weighted particle size distribution profiles of a cannabinoid comprising about 95% w/w CBD distillate;

FIG. 4A shows intensity weighted particle size distribution profiles of a cannabinoid comprising about 80% w/w CBD distillate after a stress test;

FIG. 4B shows an example of the volume weighted particle size distribution profiles of a cannabinoid comprising about 80% w/w CBD distillate after a stress test;

FIG. 5A shows intensity weighted particle size distribution profiles of a cannabinoid comprising about 80% w/w CBD distillate after another stress test;

FIG. 5B shows an example of the volume weighted particle size distribution profiles of a cannabinoid comprising about 80% w/w CBD distillate after another stress test;

FIG. 6A shows intensity weighted particle size distribution profiles of a cannabinoid associated with FIGS. 5A and 5B after another freeze-thaw cycle;

FIG. 6B shows an example of the volume weighted particle size distribution profiles of a cannabinoid associated with FIGS. 5A and 5B after another freeze-thaw cycle;

FIG. 7A shows intensity weighted particle size distribution profiles of a cannabinoid associated with FIGS. 6A and 6B after another freeze-thaw cycle;

FIG. 7B shows an example of the volume weighted particle size distribution profiles of a cannabinoid associated with FIGS. 6A and 6B after another freeze-thaw cycle;

FIG. 8A shows intensity weighted particle size distribution profiles of a cannabinoid comprising about 80% w/w CBD distillate after another stress test;

FIG. 8B shows an example of the volume weighted particle size distribution profiles of a cannabinoid comprising about 80% w/w CBD distillate after another stress test;

FIG. 9A shows intensity weighted particle size distribution profiles of a cannabinoid comprising about 80% w/w CBD distillate after another stress test; and

FIG. 9B shows an example of the volume weighted particle size distribution profiles of a cannabinoid comprising about 80% w/w CBD distillate after another stress test.

### DETAILED DESCRIPTION

An emulsion formulation that has substantial advantages over general commercially available infusion technologies is described. The formulation includes high dispersibility into water, low to no perceptible off-flavors in the diluted emulsion, remarkably high cannabinoid stability and rapid onset of effects compared to oils and other edible preparations.

Described herein is a formulation for a water-dispersible cannabinoid emulsion. In an example, the composition of a water-dispersible cannabinoid base emulsion includes a cannabinoid, cannabis-derived compounds, or compounds found in cannabis, an edible oil or partially-hydrolyzed or alcoholized edible oil, and an emulsifier. For example, a cannabinoid may include cannabidiol (CBD), cannabinol (CBN), cannabigerol (CBG), cannabichromene (CBC), cannabidivarol (CBDV), tetrahydrocannabidiol (THCBD), tetrahydrocannabigerol (THCBG), tetrahydrocannabichromene (THCBC), tetrahydrocannabidivarol (THCBDV), or derivatives thereof.

Cannabis-derived compounds may include cannabinoids, cannabinoid acids, terpenoids (such as alpha-bisabolol, alpha-cedrene, alpha-humulene, alpha-phellandrene, alpha-pinene, alpha-terpinene, alpha-terpineol, beta-caryophyllene, beta-myrcene, beta-ocimene, beta-pinene, borneol, camphene, camphor, caryophyllene oxide, cedrol, cis-nerolidol, d-limonene, delta-3-carene, e-beta-farnesene, eucalyptol, fenchol, fenchone, gamma-terpinene, geraniol, geranyl acetate, guaiol, isoborneol, isopulegol, linalool, menthol, nerol, p-cymene, pulegone, sabinene, sabinene hydrate, terpinolene, trans-nerolidol, valencene, and other isomers or analogues thereof), flavonoids, hydrocarbons, fatty acids, phenols, and others The edible oils or partially-hydrolyzed edible oil used in the composition is not particularly limited and may include corn oil, coconut oil, canola oil, olive oil, rapeseed oil, grapeseed oil, avocado oil, palm oil, palm kernel oil, rice bran oil, peanut oil, sunflower oil, castor oil, canola oil, safflower oil, flaxseed oil, vegetable oil, or other triglyceride oils, and hydrolyzed, alcoholized, partially-hydrolyzed or partially-alcoholized versions of those oils, which may contain, in varying ratios, monoglycerides, diglycerides and triglycerides derived from the edible oils. The emulsifier may be any chemical or natural additive that encourages the suspension of a liquid in another liquid where the two liquids are not soluble in each other. For example, an emulsifier may include phospholipids, saponins, pectin, gum arabic, gum acacia, modified acacia gum, chitosan, sucrose esters of fatty acids, polysorbates, sorbitan esters of fatty acids, cholesterol, acetylated monoglycerides, agar, algin, carrageenan, arabino-galactan, carob bean gum, carboxymethyl cellulose, citric acid esters of mono and diglycerides, gellan gum, guar gum, hydroxylated phospholipids, lactylated mono and diglycerides, polyglycerol esters of fatty acids, propylene glycol alginate and other propylene glycol esters.

In some other examples, the composition of a water-dispersible cannabinoid base emulsion may include an acidulant and/or a pH adjusting agent. Examples of acidulants or pH adjusting agents may include acetic acid, adipic acid, ammonium aluminum sulphate, ammonium bicarbonate, ammonium carbonate, ammonium citrate, ammonium hydroxide, ammonium phosphate, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, calcium sulphate, citric acid, cream of tartar, fumaric acid, gluconic acid, hydrochloric acid, lactic acid, magnesium carbonate, magnesium citrate, magnesium fumarate, magnesium hydroxide, magnesium oxide, magnesium sulphate, magnesium phosphate, malic acid, manganese sulphate, metatartaric acid, phosphoric acid, potassium acid tartrate, potassium aluminum sulphate, potassium bicarbonate, potassium carbonate, potassium chloride, potassium citrate, potassium fumarate, potassium hydroxide, potassium lactate, sodium acetate, sodium acid pyrophosphate, sodium acid tartrate, sodium aluminum phosphate, sodium aluminum sulphate, sodium bicarbonate, sodium bisulphate, sedum carbonate, sodium citrate, sodium fumarate, sodium gluconate, sodium hexametaphosphate, sodium hydroxide, sodium lactate, sodium phosphate, sodium potassium hexametaphosphate, sodium potassium tartrate, sodium potassium tripolyphosphate, sodium pyrophosphate, sulphuric acid, sulphurous acid, tartaric acid, and others. The amount of acidulant that may be added may depend on various factors such as the acidulant used to achieve a target taste and/or end use for the emulsion.

In some other examples, the composition of a water-dispersible cannabinoid base emulsion may include a citrus oil or other flavoring agents. Examples of citrus oils may include lemon oil, orange oil, grapefruit oil, sweet orange oil, bitter orange oil, blood orange oil, lime oil, bergamot oil, mandarin oil, tangerine oil, yuzu oil, petitgrain oil, neroli oil, and others. The amount of citrus oil that may be added may depend on various factors such as the citrus oil used and desired flavor. As a specific example, lemon oil concentrations of about 0.5% to about 1% will produce a fresh citrus aroma and mild flavor. Concentrations between about 1% and about 4% will produce a strong flavor and aroma. Examples of flavoring agents may include other essential oils, for example; anise oil, basil oil, black pepper oil, cannabis flower essential oil, caraway seed oil, lavender oil, peppermint oil, the chemical equivalents of those essential oils or combinations of essential oils, and artificial flavoring agents that are of synthetic origin but formulated to imitate an essential oil, combination of essential oils, or a produce a perceived flavor when consumed. The amount of flavoring agents that may be added may depend on various factors such as the desired flavor. As a specific example, if a lemon-lime flavor is desired, lemon essential oil and lime essential oil can be added so that the sum of ingredients are in the range of about 0.5% to about 4%.

In another example of a composition for a water-dispersible cannabinoid base emulsion, the composition may include one or more of:
a) Polysorbate 80 at between about 0.1% to about 5% by weight; preferably, between about 0.5% and about 2% by weight.
b) Sorbitan monostearate at between about 0.1% to about 5% by weight; preferably, between about 0.5% and about 2% by weight.
c) Corn oil mono and diglycerides at between about 0.1% to about 15% by weight; preferably, between about 1% and about 10% by weight.
d) Cannabinoid, such as tetrahydrocannabinol (THC) or cannabidiol (CBD) at between about 0.1% to about 5% by weight; preferably, between about 0.5% and about 3% by weight, or alternatively, between about 0.5% and about 5% by weight.
e) Lemon oil at between about 0.1% to about 6% by weight; preferably, between about 0.5% and about 3% by weight.
f) Citric acid at between about 0.1% to about 8% by weight; preferably, between about 0.5% and about 2% by weight.
g) Potassium citrate at between about 0.1% to about 6% by weight; preferably, between about 0.5% and about 3% by weight. and
h) Water at between about 20% to about 95% by weight.

Polysorbate 80 is a non-ionic surfactant that is used as an excipient in drug formulation and as an emulsifier in food and cosmetic products (E433). It is commercially available from suppliers such as BASF (trade name Kolliphor PS 80), Croda (trade name Tween 80), and others.

Sorbitan monostearate is a non-ionic surfactant that is used as an excipient in food and healthcare products for the purpose of thickening, emulsifying, dispersing, and wetting (E491). It is commercially available from suppliers such as Croda (Span 60) and others.

Corn oil mono and diglycerides are mixtures of mono, di, and triglycerides of long chain fatty acids derived by partial hydrolysis or alcoholysis of corn oil. It is commercially available from suppliers such as Gattefosse (Maisine CC) or others.

Cannabinoids are molecules found in the cannabis plant (or synthetic equivalents) that interact with the endocannabinoid system, a biological system composed of cannabinoid receptors (e.g. CB1 and CB2) that are expressed throughout the central and peripheral nervous systems of vertebrates. Some of these compounds are extracted or physically separated from Cannabis Sativa and can be purified by distillation to produce a cannabinoid distillate (e.g. THC distillate) and/or recrystallization to produce a cannabinoid isolate (e.g. CBD isolate). Due to the variations in starting materials, which are of biological origin and thus highly susceptible to variations in concentrations of cannabinoids, the purities of a given cannabinoid in a distillate or in an isolate may vary and may be expressed as a percentage of mass e.g. 80% THC distillate).

Lemon oil is an essential oil extracted from Citrus Limon. It is widely used as a flavoring agent in food and beverage products, as a fragrance in personal care products and consumer products more generally. The primary compound in lemon oil is limonene, a monoterpene that may be derived from natural sources (e.g. citrus plants and their fruits, many types of coniferous and broadleaved trees) or industrially produced through chemical synthesis or biosynthesis. It is commercially available from suppliers such as Jedwards International, Vigon, and others.

Citric acid is an organic acid found naturally in many fruits and vegetables, especially in citrus fruits. Citric acid is also produced industrially through the fermentation of sugars in the presence of various strains of aspergillus niger. It is used as an acidulant, preservative, emulsifying agent, and chelating agent, among other applications, in food products, beverage products, topical products, cosmetic products, and consumer goods in general.

In another example of a composition for a water-dispersible cannabinoid base emulsion, the composition may include one or more of:
a) Polysorbate 80 at about 1.00% by weight;
b) Sorbitan monostearate at about 1.00% by weight;
c) Corn oil mono and diglycerides at about 1.95% by weight;
d) Cannabinoid, such as tetrahydrocannabinol (THC) or cannabidiol (CBD) at about 2.00% by weight;
e) Lemon oil at about 0.50% by weight;
f) Citric acid at about 0.26% by weight;
g) Potassium citrate at about 0.26% by weight; and
h) Water at about 93.03% by weight.

In another example, the composition of the water-dispersible cannabinoid base emulsion may be modified to include additional preservatives. The amount and type of preservatives that may be added may depend on various factors such as the intended use of the emulsion and the desired shelf life under various conditions as well as other factors. It is to be appreciated by a person of skill in the art with the benefit of this description that the acidulants and preservative concentrations may be adjusted in tandem to achieve a target pH range and to enhance microbial resistance. The increased microbial resistance may be useful in non-sterile manufacturing environments or food processing plants where microbes may be encountered. Increased microbial resistance may also be useful in applications where products are stored and consumed in resealable or reusable containers or bags.

In this example of a composition for a water-dispersible cannabinoid base emulsion, the composition may include one or more of:
a) Polysorbate 80 at about 1.00% by weight;
b) Sorbitan monostearate at about 1.00% by weight;
c) Corn oil mono and diglycerides at about 1.95% by weight;
d) Cannabinoid, such as tetrahydrocannabinol (THC) or cannabidiol (CBD) at about 2.00% by weight;
e) Lemon oil at about 0.50% by weight;
f) Citric acid at about 0.50% by weight;
g) Potassium citrate at about 0.10% by weight;
h) Potassium sorbate at about 0.04% by weight; and
i) Water at about 93% by weight.

It is understood by a person of skill with the benefit of this description that the amount of cannabinoid added to achieve a desired therapeutic or recreational result is influenced by, and will therefore vary based on, a number of factors, including for example and without limitation, the age, sex, and weight of the subject, factors that influence the metabolic rate, and other characteristics of the subject. The concentration of at least one cannabinoid in compositions provided herein is between about 0.1% to about 5%.

In another example, the cannabinoid concentrations of the water-dispersible cannabinoid base emulsion may be increased proportionally with surfactants and oils to maintain consistent emulsion colloidal properties and flavor.

In another example of a composition for a water-dispersible cannabinoid base emulsion, the composition may include one or more of:
a) Polysorbate 80 at about 2.00% by weight;
b) Sorbitan monostearate at about 2.00% by weight;
c) Corn oil mono and diglycerides at about 3.90% by weight;
d) Cannabinoid, such as tetrahydrocannabinol (THC) or cannabidiol (CBD) at about 4.00% by weight;
e) Lemon oil at about 1.00% by weight;
f) Citric acid at about 0.50% by weight;
g) Potassium citrate at about 0.10% by weight;
h) Potassium sorbate at about 0.04% by weight; and
i) Water at about 86% by weight.

A person of skill in the art with the benefit of this description will understand that the ingredients in the final formulations are to total 100%, and based on the teachings provided herein, will understand that modifications to the exemplary formulations provided herein are possible (e.g., replacement of a recited ingredient with a different ingredient, addition of a different ingredient, and/or modification of an amount of an ingredient) provided that such modifications result in a formulation as taught and described herein (i.e., capable of delivering an active agent such as a cannabinoid topically).

The creation of a base emulsion, such as a base emulsion described above or a variation described in more detail below is not particularly limited. For example, the process may involve preparing a buffer solution with about 0.26% w/w citric acid and about 0.26% w/w potassium citrate in water. An aqueous surfactant solution is then prepared by dispersing about 1.00% w/w polysorbate 80 and about 1.00% w/w sorbitan monostearate in the buffer solution and heating it to about 65° C. with continuous stirring. Next, about 1.95% w/w corn oil mono and diglycerides is heated to about 65° C., and about 0.5% w/w lemon oil and about 2% w/w cannabinoid were added and mixed to generate a lipid phase. An oil-in-water pre-emulsion is obtained by combining the hot aqueous surfactant solution and the hot lipid phase by high shear mixing with a rotor-stator mixer. High-pressure homogenization using Microfluidizer® is carried out in a pre-heated system (about 60° C.) at about 30 Kpsi for 3 cycles; the outlet temperature was adjusted to about 15° C. The resulting emulsion is then stored at a temperature between about 0° C. to about 4° C.

In other examples, the creation of the base emulsion may be carried out using other mixing methods. For example, a high-shear homogenization method may be used to generate the water-dispersible cannabinoid base emulsion. In another example, the water-dispersible cannabinoid base emulsion may be created using ultrasonication or with ultrasonic processing. In another example, the water-dispersible cannabinoid base emulsion may be created using solvent emulsification and/or evaporation methods.

Dynamic light scattering is a widely used method for evaluating the physicochemical properties of colloidal systems, including emulsions. The method involves the measurement of particle-scattered light intensities over time due to diffusion of particles in a sample. Thus, information such as the diffusion coefficients of the particles can be determined and the particle size distributions, and other physicochemical properties can also be determined. Since changes in particle size distributions in an emulsion can result in changes to the emulsion's stability over time, DLS measurements of an emulsion over time can provide insights into the colloidal stability of the emulsion. Emulsions that are resistant to instability mechanisms like Ostwald ripening, creaming, sedimentation, flocculation, coalescence, and ultimately phase separation can be selected by visual observation of the sample over time and by measurement of a sample's particle size distributions over time using dynamic light scattering techniques.

In order to determine the emulsions' average particle sizes (dz), polydispersity indices (PDI) and zeta potentials, dynamic light scattering (DLS) also known as photon correlation spectroscopy, was used. The measurements were conducted using a Malvern Zetasizer (Nano ZS, Malvern Instruments Ltd., UK). The supplier's software (Zetasizer Software 7.13) was used to analyze the data and derive the average particle sizes, polydispersity indices and zeta potentials for the emulsion samples.

The base emulsion formulations produced with slightly different cannabinoid-containing raw materials (e.g. distillates or isolates) may have slightly different physicochemical properties. For example, the base emulsion prepared with CBD isolate rather than CBD distillate may have smaller particle sizes if the viscosity of the emulsion's oil phase more closely matches that of the emulsion's aqueous phase. In one instance, the base emulsion produced using the same processing methods but with different cannabinoid-containing raw materials produced slightly different average particle sizes (dz), polydispersity indices (PDI) and zeta potentials. In the present example, the average particle sizes of the emulsions produced with sufficiently high purity raw materials, such as greater than about 80% pure by weight, may be in the range of about 200 nm to about 800 nm. Exemplary results are outlined below in Table 1:

TABLE 1

| Cannabinoid | Average Particle | Polydispersity | Zeta Potential |
|---|---|---|---|
| Raw Material | Size (nm) | Index | (mV) |
| 85% THC distillate | 674 ± 14 | 0.25 | −8.9 |
| 80% CBD distillate | 202 ± 3 | 0.18 | −8.4 |
| 95% CBD isolate | 591 ± 6.7 | 0.391 | −12.3 |

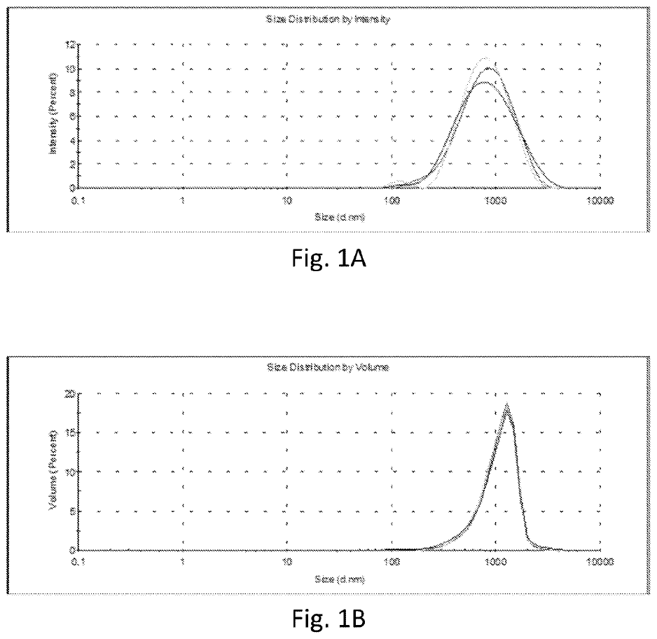

Referring to FIGS. 1A and 1B characterizations of a base emulsion created with a cannabinoid comprising about 85% w/w THC distillate.

FIG. 1A shows an example of the intensity weighted particle size distribution profiles obtained from a dynamic light scattering experiment performed in triplicate using a Malvern Zetasizer.

FIG. 1B shows an example of the volume weighted particle size distribution profiles obtained from a dynamic light scattering experiment performed in triplicate using a Malvern Zetasizer.

In this example, the average size of the droplets was determined to be about 674 nm±14 nm and the polydispersity index for this example was determined to be about 0.25. The average zeta potential for this example base emulsion was measured to be about-8.9 mV.

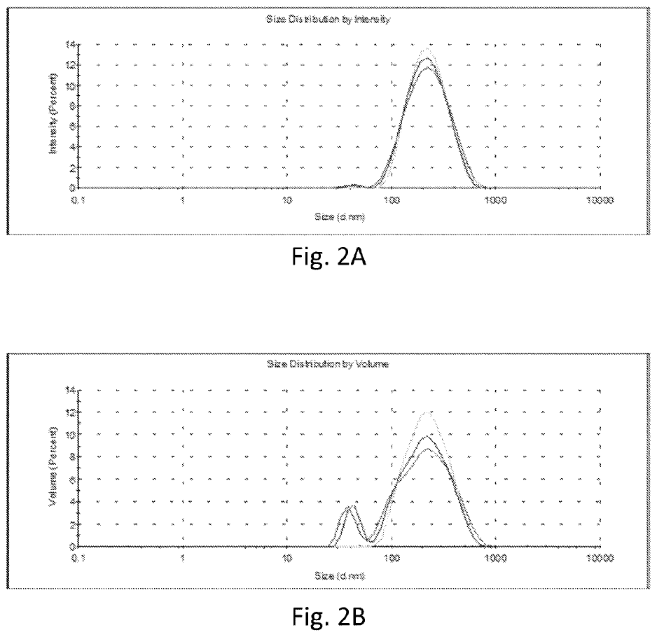

Referring to FIGS. 2A and 2B, characterizations of another base emulsion created with a cannabinoid comprising about 80% w/w CBD distillate.

FIG. 2A shows an example of the intensity weighted particle size distribution profiles obtained from a dynamic light scattering experiment performed in triplicate using a Malvern Zetasizer.

FIG. 2B shows an example of the volume weighted particle size distribution profiles obtained from a dynamic light scattering experiment performed in triplicate using a Malvern Zetasizer.

In this example, the average size of the droplets was determined to be about 202 nm±3 nm and the polydispersity index for this example was calculated to be about 0.18. The average zeta potential for this example base emulsion was measured to be about-8.4 mV.

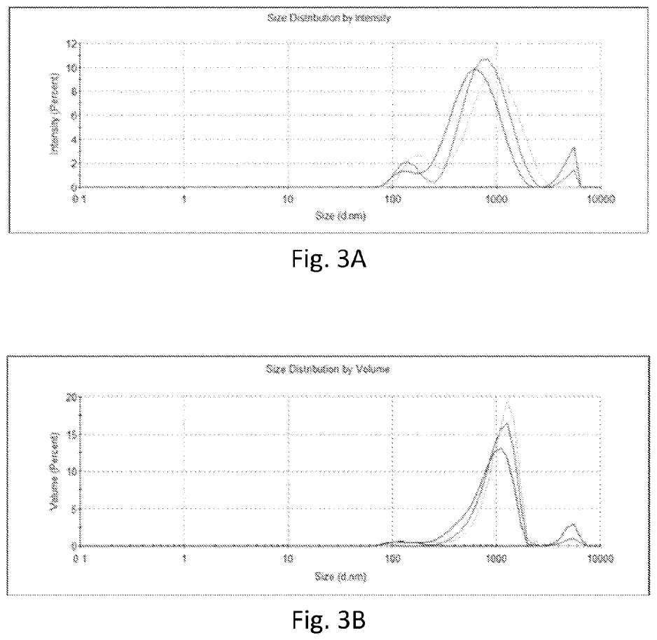

Referring to FIGS. 3A and 3B, characterizations of another base emulsion created with a cannabinoid comprising about 95% w/w CBD distillate.

FIG. 3A shows an example of the intensity weighted particle size distribution profiles obtained from a dynamic light scattering experiment performed in triplicate using a Malvern Zetasizer.

FIG. 3B shows an example of the volume weighted particle size distribution profiles obtained from a dynamic light scattering experiment performed in triplicate using a Malvern Zetasizer.

In this example, the average size of the droplets was determined to be about 591 nm±6.7 nm and the polydispersity index for this example was calculated to be about 0.391. The average zeta potential for this example base emulsion was measured to be about-12.3 mV.

When using different cannabinoid input materials, several factors can affect the properties of the finished base emulsion. For example, some distillates with higher THC concentrations have higher viscosities, which may result in a more viscous dispersed phase in the base emulsion, which may in turn cause average droplet sizes to be slightly larger. Conversely, some distillates with lower cannabinoid potencies and/or higher total terpenoid concentrations may have lower viscosities, which may result in a less viscous dispersed phase in the base emulsion, which may in turn cause average droplet sizes to be slightly smaller. Additionally, this may also cause the emulsion to have an acrid, bitter, or cannabis-like aroma, odor or flavor, which may be desired or undesired depending on finished product attributes.

In an example, the stability of the base emulsion formulation with about 80% CBD distillate was assessed by subjecting the emulsion to several stress tests. These tests mimic some potential conditions that the emulsion or products produced using the emulsion as an ingredient might face during the production of a cannabis infused product, for example, a cannabis infused beverage.

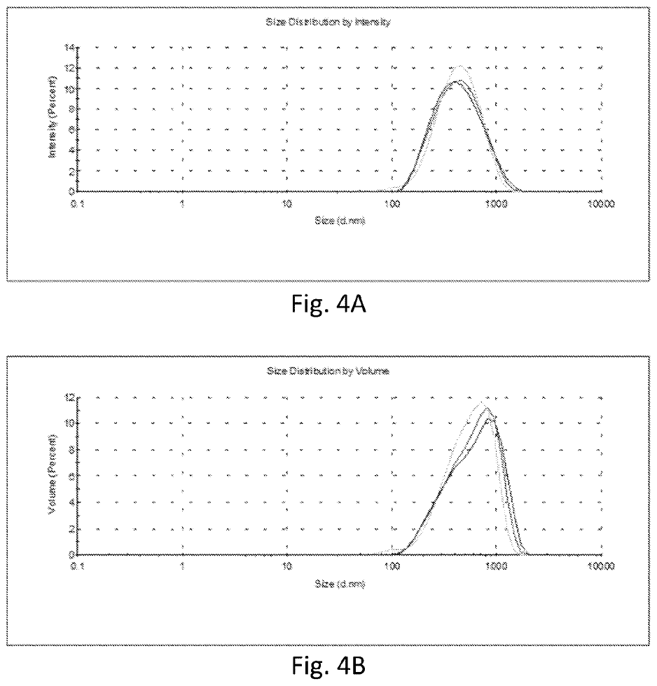

Referring to FIGS. 4A and 4B, results from a stress test of the base emulsion measured in triplicate is shown. The stress test is carried out with a cannabinoid base emulsion comprising about 80% w/w CBD distillate. In the present example, the base emulsion described in association with FIGS. 2A and 2B was flash heated to test its stability. The stress test involved placing about 1 g of the base emulsion in a preheated water bath. The water bath was maintained at about 80° C. to hold the internal temperature of the nanoemulsion at about 80° C. for about 1 minute. This protocol is a more extreme version of the high-temperature short-time (HTST) pasteurization process that fruit juices and milk beverages are subjected to in the beverage industry, which may involve maintaining a sample at about 71.5° C. for about 15 s. The base emulsion was then allowed to cool to about 23° C. and a part of the base emulsion was diluted about 1:50 for dynamic light scattering analysis to generate the graphs in FIGS. 4A and 4B.

Based on visual observations, the pasteurized base emulsion showed no visible signs of creaming, flocculation, sedimentation or coalescence. In this example, the average size of the droplets was increased to about 378 nm±3.4 nm. The polydispersity index for this example is calculated to be about 0.18, which remained relatively unchanged. The average zeta potential for this example base emulsion dropped to about −18.3 mV. Overall, these results show that the stability of the base emulsion after the stress test is reasonably maintained, which indicates that the base emulsion is stable and predicted to have a long self-life even when exposed to higher than ambient temperatures, such as during processing, pasteurization, transportation or storage.

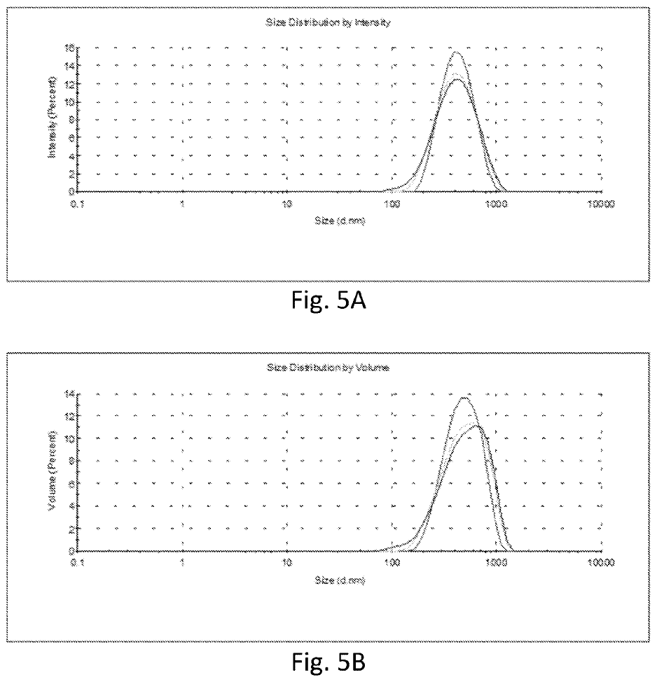

Referring to FIGS. 5A and 5B, results from another stress test of the base emulsion measured again in triplicate is shown. The stress test is carried out with a cannabinoid base emulsion comprising about 80% w/w CBD distillate. In the present example, the base emulsion described in association with FIGS. 2A and 2B was subjected to a freeze-thaw cycle to test its stability. In particular, about 1 g of the emulsion was placed in a freezer at a temperature of about-20° C. for about 1 hour. The sample of the base emulsion was removed from the freezer and allowed to revert to room temperature. A part of the thawed emulsion was diluted for dynamic light scattering analysis to generate the graphs in FIGS. 5A and 5B. The emulsion showed moderate stability under freeze thaw cycling and maintained water-dispersibility after freeze/thaw cycles.

In this example, the average size of the droplets was determined to be about 373 nm±3.3 nm based and the polydispersity index for this example was calculated to be about 0.16. The average zeta potential for this example base emulsion was measured to be about-16.7 mV.

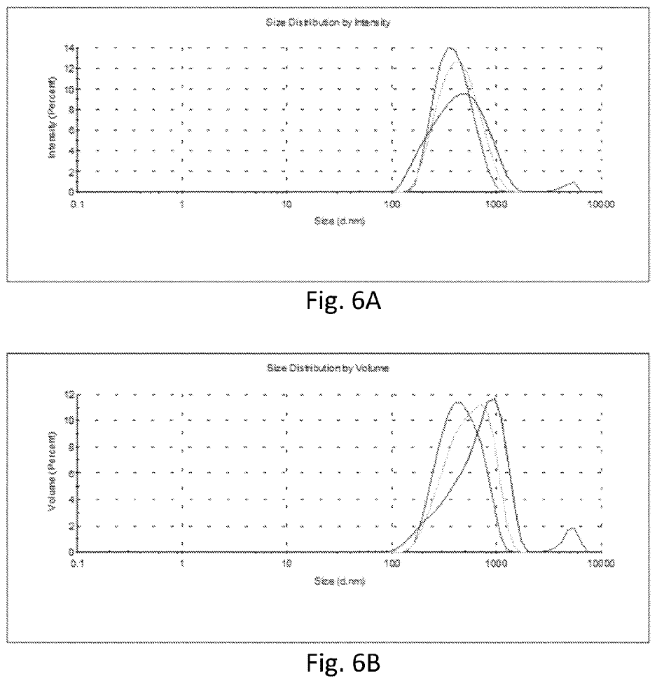

Referring to FIGS. 6A and 6B, results from another freeze-thaw cycle of the base emulsion shown in FIGS. 5A and 5B measured in triplicate is shown. In the present example, the freeze-thaw cycle involved placing the emulsion in a freezer at a temperature of about-20° C. for about 1 hour. The sample of the base emulsion was removed from the freezer and allowed to revert to about 23° C. Accordingly the results shown in FIGS. 6A and 6B have been subjected to two substantially similar freeze-thaw cycles.

After the second freeze-thaw cycle, the average size of the droplets was determined to be about 377 nm±2.6 nm and the polydispersity index for this example was calculated to be about 0.20. The average zeta potential for this example was measured to be about-16.6 mV. Accordingly, the second freeze-thaw cycle did not significantly change the properties of the base emulsion.

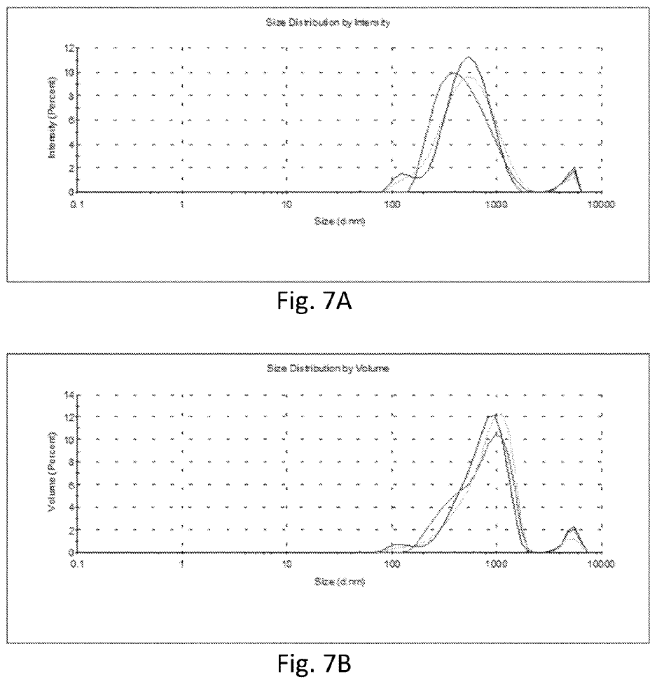

Referring to FIGS. 7A and 7B, results from another freeze-thaw cycle of the base emulsion shown in FIGS. 6A and 6B measured in triplicate is shown. In the present example, the freeze-thaw cycle involved placing the emulsion in a freezer at a temperature of about-20° C. for about 1 hour. The sample of the base emulsion was removed from the freezer and allowed to revert to about 23° C. Accordingly the results shown in FIGS. 7A and 7B have been subjected to three substantially similar freeze-thaw cycles.

After the third freeze-thaw cycle, the average size of the droplets increased to about 465 nm±19.5 nm based and the polydispersity index for this example was calculated to have increased to about 0.32. The average zeta potential for this example base emulsion was measured to be about-15.8 mV.

As can be seen by this example, minor changes in the particle size distributions may occur upon freezing and thawing of the base emulsion, and a trend of increasing polydispersity and average particle size is apparent after each cycle. Thus, in idealized conditions, the emulsion should not be subjected to several freeze that cycles before use as intended.

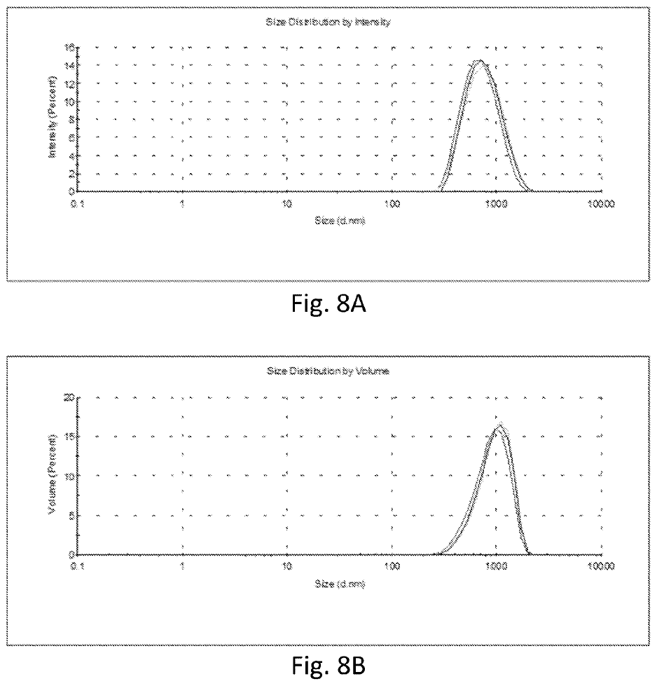

Referring to FIGS. 8A and 8B, results from another stress test of the base emulsion measured in triplicate is shown. The stress test is carried out with a cannabinoid base emulsion comprising about 80% w/w CBD distillate. In the present example, the base emulsion described in association with FIGS. 2A and 2B was subjected to dilution and carbonation to test its stability for use in some beverages. In particular, about 1 mL of the base emulsion was diluted into about 355 mL of carbonated water. The diluted emulsion was then subjected to dynamic light scattering measurements.

Based on visual observations, the diluted and carbonated emulsion showed no signs of creaming, flocculation, sedimentation or coalescence. In this example, the average size of the droplets was determined to be about 669 nm±19.8 nm by DLS. The polydispersity index for this example was calculated to be about 0.15. The average zeta potential for this example was measured to be about −26.7 mV. There was substantially no change to the particle size distributions upon dilution and carbonation.

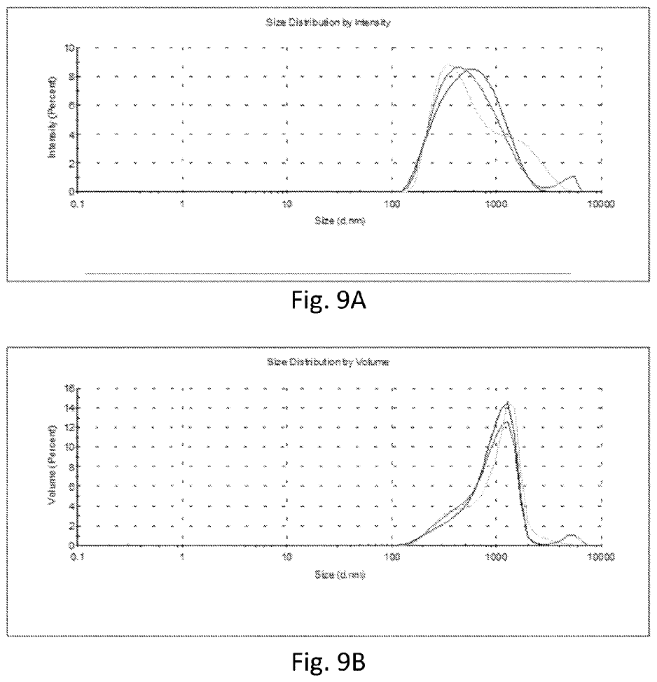

Referring to FIGS. 9A and 9B, results from another stress test of the base emulsion measured in triplicate is shown. The stress test is carried out with a cannabinoid base emulsion comprising about 80% w/w CBD distillate. In the present example, the base emulsion described in association with FIGS. 2A and 2B was subjected to acidulation to test its stability. In particular, dilute hydrochloric acid (0.1 mM) was added dropwise to about 1 mL of emulsion until the pH reached about 3.0. Dilution for dynamic light scattering analysis was performed with a pH 3 buffer solution.

Based on visual observations, the acidulated emulsion showed no visible signs of creaming, flocculation, sedimentation or coalescence. In this example, the average size of the droplets was determined to be about 483 nm±4.1 nm and the polydispersity index for this example was calculated to be about 0.27. The average zeta potential for this example was measured to be about −16.1 mV. There was substantially no change to the particle size distributions occur upon acidulation.

Referring to table 2, observations of certain properties of the emulsions comprising various cannabinoid-containing input materials were tracked over time after manufacturing. The emulsions were stored in a dark fridge at about 4° C. in sealed HDPE containers and sampled periodically as outlined in table 2. In the present example, the appearance, cannabinoid concentrations, and pH were measured and recorded over time to demonstrate that the emulsions are chemically stable. In addition, a microbiological scan was carried out by a third-party lab periodically over the course of several months. The microbiological scan tested for the presence of bacteria, yeast and moulds in the samples which may indicate spoilage.

TABLE 2

| | | | THC | CBD | | | |
|---|---|---|---|---|---|---|---|
| Cannabinoid | | | Potency | Potency | | Micro | |
| Ingredient | Time | | (mg/g) | (mg/g) | pH | Scan | Appearance |
| 95% CBD | Initial | | 0 | 22.3 | 4.03 | None | Smooth, opaque |
| isolate | | | | | | detected | white liquid. |
| | 1 | Month | 0 | 22.0 | 3.98 | None | Unchanged |
| | | | | | | detected | |
| | 2 | Month | 0 | 22.0 | — | — | Unchanged |
| | 3 | Month | | 22.2 | 4.04 | None | Unchanged |
| | | | | | | detected | |
| | 4 | Month | 0 | 22.0 | — | — | Unchanged |
| | 5 | Month | 0 | 21.5 | — | — | Unchanged |
| | 6 | Month | 0 | 20.8 | — | — | Unchanged |
| 85% THC | Initial | | 19.3 | 0 | 4.04 | None | Smooth, opaque |
| distillate | | | | | | detected | white liquid. |
| | 1 | Month | 18.9 | 0 | 4.03 | None | Unchanged |
| | | | | | | detected | |
| | 2 | Month | 19.2 | 0 | — | — | |
| | 3 | Month | 19.5 | 0 | 4.03 | None | Unchanged |
| | | | | | | detected | |
| | 4 | Month | 18.6 | 0 | — | — | Unchanged |
| | 5 | Month | 19.0 | 0 | — | — | Unchanged |
| | 6 | Month | 19.7 | 0 | 4.03 | None | Unchanged |
| | | | | | | detected | |
| | 7 | Month | 19.3 | 0 | — | — | Unchanged |
| | 9 | Month | 18.9 | 0 | — | — | Unchanged |
| | 12 | Month | 18.0 | 0 | 4.02 | None | Unchanged |
| | | | | | | detected | |

A series of variations to the base formulation were prepared to better understand the relationship between ingredient ratios, and emulsion physicochemical properties produced under the same or similar processing conditions

Referring to table 3, Var 9 displayed visually detectable colloidal destabilization after manufacturing. In particular, creaming of the emulsion was detected where oil builds up on the surface of the emulsion. It is to be understood by a person of skill with the benefit of this description that as long as the particle size distributions of the emulsion remain substantially Gaussian, the average particle sizes stay below about 1.0 micron or close to the initial values, the emulsion polydispersity index stays relatively tight (<0.3) or unchanged, and the visual inspections of the emulsions don't show any signs of creaming, flocculation, sedimentation, etc., the emulsions may be considered stable. Accordingly, the results indicate that certain variations of the base formulation may produce emulsions with acceptable stability for use in manufacturing of cannabinoid-containing products.

TABLE 3

| | | w/w % corn | | | | D_{z} | | |
|---|---|---|---|---|---|---|---|---|
| | w/w % | oil mono | | | | (nm) | PDI | Span |
| Emulsion | polysorbate | and | D_{z} | | | after 2 | after 2 | after 2 |
| formulation | 80 | diglycerides | (nm) | PDI | Span | weeks | weeks | weeks |
| Base | 1 | 1.95 | 202 | 0.18 | 1.6 | 124 | 0.24 | 2.2 |
| Var 2 | 1 | 3.95 | 195 | 0.16 | 1.3 | 212 | 0.23 | 8.7 |
| Var 3 | 1 | 5.95 | 143 | 0.11 | 1.2 | 407 | 0.49 | 3.0 |
| Var 4 | 2 | 1.95 | 152 | 0.14 | 1.4 | 147 | 0.15 | 1.5 |
| Var 5 | 2 | 3.95 | 206 | 0.23 | 2.1 | 159 | 0.16 | 1.5 |
| Var 6 | 2 | 5.95 | 169 | 0.12 | 1.2 | 172 | 0.14 | 1.4 |
| Var 7 | 3 | 1.95 | 93 | 0.22 | 1.6 | 99 | 0.24 | 1.8 |
| Var 8 | 3 | 3.95 | 132 | 0.19 | 1.9 | 127 | 0.18 | 1.5 |
| Var 9 | 3 | 5.95 | 208 | 0.41 | 21.3 | 218 | 0.47 | 21.9 |

The base emulsions described above may be used in a variety of consumable products such as beverages. Several beverages were compounded with the base emulsion and stored in cans or bottles at ambient temperature and conditions to measure the cannabinoid concentrations over time. Referring to table 4, properties such as the cannabinoid concentration, the beverage pH, dissolved gasses and Brix at different timepoints are outlined below.

The results show that many types of cannabinoid-infused beverages with excellent shelf life can be prepared using the base emulsion.

TABLE 4

| | | | THC | CBD | | | | Carbon- | Dissolved |
|---|---|---|---|---|---|---|---|---|---|
| Beverage | | | Potency | Potency | | Brix | Density | ation | Oxygen |
| Type | Time | | (mg/g) | (mg/g) | pH | (°Bx) | (g/mL) | (vol) | (ppb) |
| Canned | Initial | | 0 | 0.032 | 3.40 | 0.32 | 0.999 | 2.67 | 973 |
| Grapefruit | 1 | Month | 0 | 0.029 | 3.51 | 0.33 | 0.999 | 2.79 | 1926 |
| Seltzer | 2 | Month | 0 | 0.030 | 3.68 | 0.33 | 0.999 | 2.55 | 2089 |
| | 3 | Month | 0 | 0.030 | 3.6 | 0.31 | 0.999 | 2.57 | 2019 |
| Canned | Initial | | 0.030 | 0 | 3.24 | 4.80 | 1.017 | 2.28 | 2478 |
| Carbonated | 1 | Month | 0.029 | 0 | 3.57 | 4.84 | 1.017 | 2.45 | 253 |
| White Wine | 2 | Month | 0.029 | 0 | 3.57 | 4.84 | 1.017 | 2.46 | 253 |
| | 3 | Month | 0.031 | 0 | 3.64 | 4.9 | 1.017 | 3.1 | 20.4 |
| | 6 | Month | 0.029 | 0 | 3.64 | 4.88 | 1.017 | 2.97 | 23.1 |
| | 9 | Month | 0.030 | 0 | — | — | — | — | — |
| | 12 | Month | 0.030 | 0 | 3.63 | 4.79 | 1.017 | 2.17 | 67 |
| Bottled | Initial | | 0.029 | 0 | 3.30 | 4.53 | 1.017 | 0.01 | 1991 |
| White Wine | 1 | Month | 0.029 | 0 | 3.54 | 5.43 | 1.016 | 0.01 | 313 |
| | 2 | Month | 0.029 | 0 | 3.54 | 4.53 | 1.016 | 0.01 | 313 |
| | 3 | Month | 0.030 | 0 | 3.65 | 4.53 | 1.016 | 0.01 | 363 |
| | 6 | Month | 0.029 | 0 | 3.55 | 4.53 | 1.016 | 0.02 | 299 |
| | 9 | Month | 0.029 | — | — | — | — | — | — |
| | 12 | Month | 0.030 | 0 | 3.59 | 4.54 | 1.016 | 0.05 | 265 |
| Canned | Initial | | 0.011 | 0 | 3.18 | 9.76 | 1.037 | 1.9 | 687 |
| Cola | 1 | Month | 0.011 | 0 | 3.23 | 9.8 | 1.037 | 1.8 | 1324 |
| | 2 | Month | 0.010 | 0 | 3.24 | 9.83 | 1.037 | 1.63 | 725 |
| | 3 | Month | 0.009 | 0 | 3.30 | 9.81 | 1.037 | 1.87 | 480 |
| Canned | Initial | | 0.029 | 0 | 2.96 | 4.41 | 1.015 | 2.52 | 484 |
| Margarita | 1 | Month | 0.029 | 0 | — | — | — | — | — |
| | 2 | Month | 0.028 | 0 | — | — | — | — | — |
| | 3 | Month | 0.028 | 0 | 3.01 | 4.51 | 1.016 | 2.87 | 24.8 |
| | 6 | Month | 0.029 | 0 | — | — | — | — | — |
| Canned | Initial | | 0.013 | 0 | 3.05 | 8.43 | 1.032 | 1.98 | 332 |
| Orange | 1 | Month | 0.013 | 0 | 3.14 | 8.51 | 1.032 | 1.9 | 112 |
| Soda | 2 | Month | 0.013 | 0 | — | — | — | — | — |
| | 3 | Month | 0.013 | 0 | 3.08 | 8.68 | 1.033 | 2.16 | 89.7 |
| | 6 | Month | 0.013 | 0 | 3.07 | 8.73 | 1.033 | 2.12 | 67.2 |
| | 9 | Month | 0.013 | 0 | 3.00 | 8.77 | 1.033 | 2.218 | 10.3 |
| Canned | Initial | | 0.028 | 0.015 | 3.47 | 9.29 | 1.035 | 2.52 | 593 |
| Tropical | 1 | Month | 0.027 | 0.014 | — | — | — | — | — |
| Punch | 2 | Month | 0.027 | 0.04 | — | — | — | — | — |
| Soda | 3 | Month | 0.027 | 0.014 | 3.44 | 9.31 | 1.035 | 2.51 | 13.5 |
| | 6 | Month | 0.029 | 0.015 | — | — | — | — | — |
| | 9 | Month | 0.027 | 0.014 | — | — | — | — | — |
| Canned | Initial | | 0.026 | 0 | 3.30 | 5.4 | 1.019 | 0.4 | 255 |
| Caesar | 1 | Month | 0.026 | 0 | — | — | — | — | — |
| | 2 | Month | 0.026 | 0 | — | — | — | — | — |
| | 3 | Month | 0.027 | 0 | 3.55 | 5.43 | 1.020 | 0.02 | 126 |
| | 6 | Month | 0.026 | 0 | 3.73 | 4.64 | 1.016 | — | — |
| | 9 | Month | 0.027 | 0 | — | — | — | — | — |
| Canned | Initial | | 0.031 | 0 | 3.64 | 1.29 | 1.003 | 2.36 | 1618 |
| Moscow | 1 | Month | 0.030 | 0 | 3.51 | 1.31 | 1.003 | 2.52 | 33.4 |
| Mule | 2 | Month | 0.028 | 0 | 3.46 | 1.31 | 1.003 | 2.53 | 12.6 |
| | 3 | Month | 0.031 | 0 | 3.54 | 1.3 | 1.003 | 2.19 | 74.5 |

It should be recognized that features and aspects of the various examples provided above may be combined into further examples that also fall within the scope of the present disclosure.

1. A water dispersible emulsion composition comprising:
a cannabinoid at about 0.1-5.0% (w/w) collectively;
an edible oil at about 0.1-15.0% (w/w); and
an emulsifying agent at about 0.1-10.0% (w/w).

2. The water dispersible emulsion composition of claim 1, wherein the cannabinoid is at about 2.00%.

3. The water dispersible emulsion composition of claim 1, wherein the cannabinoid is an 85% THC distillate.

4. The water dispersible emulsion composition of claim 1, wherein the cannabinoid is an 80% CBD distillate.

5. The water dispersible emulsion composition of claim 1, wherein the cannabinoid is a 95% CBD distillate.

6. The water dispersible emulsion composition of claim 1, wherein the edible oil comprises corn oil monoglycerides and diglycerides.

7. The water dispersible emulsion composition of claim 6, wherein the corn oil monoglycerides and diglycerides is at about 1.95%.

8. The water dispersible emulsion composition of claim 1, wherein the emulsifying agent comprises polysorbate 80.

9. The water dispersible emulsion composition of claim 8, wherein the polysorbate 80 is at about 1.00%.

10. The water dispersible emulsion composition of claim 1, wherein the emulsifying agent comprises sorbitan monostearate.

11. The water dispersible emulsion composition of claim 10, wherein the sorbitan monostearate is at about 1.00%.

12. The water dispersible emulsion composition of claim 1, further comprising an acidulant.

13. The water dispersible emulsion composition of claim 1, further comprising a pH adjusting agent.

14. (canceled)

15. The water dispersible emulsion composition of claim 1, further comprising a citric acid.

16. The water dispersible emulsion composition of claim 15, wherein the citric acid is at about 0.1-2.00%.

17. The water dispersible emulsion composition of claim 16, wherein the citric acid is at about 0.26%.

18. The water dispersible emulsion composition of claim 1, further comprising a flavoring.

19. The water dispersible emulsion composition of claim 18, wherein the flavoring is lemon oil at about 0.50%.

20. A method of preparing the water dispersible emulsion of claim 1.
